(19) United States
(12) Patent Application Publication
(10) Pub. No.: US 2026/0100278 A1
(43) Pub. Date: Apr. 9, 2026

(54) MEDICAL ASSISTANCE APPARATUS, MEDICAL ASSISTANCE SYSTEM, AND METHOD OF OPERATING MEDICAL ASSISTANCE APPARATUS

(71) Applicant: OLYMPUS MEDICAL SYSTEMS CORP., Tokyo (JP)

(72) Inventors: Megumi NAGASAWA, Tokyo (JP); Ryosuke SAKAI, Tokyo (JP); Haruhiko SAKAYORI, Kanagawa (JP); Taishi KAKEMIZU, Tokyo (JP)

(73) Assignee: OLYMPUS MEDICAL SYSTEMS CORP., Tokyo (JP)

(21) Appl. No.: 19/416,535

(22) Filed: Dec. 11, 2025

Related U.S. Application Data
(63) Continuation of application No. PCT/JP2023/022380, filed on Jun. 16, 2023.

(51) Int. Cl. G16H 40/67; A61B 1/00; A61B 1/05

(52) U.S. Cl. CPC G16H 40/67; A61B 1/00006; A61B 1/000096; A61B 1/05

(57) ABSTRACT

A medical assistance apparatus includes a processor including hardware. The processor is configured to: acquire usage status information related to a usage status of an endoscope; determine, based on the usage status information, whether or not the endoscope has been inserted into a body cavity or whether or not the endoscope has reached a desired region to be observed; when it is determined that the endoscope has not been inserted into a body cavity or that the endoscope has not reached the desired region to be observed, generate handover information including each condition and handover content in association with each other for a report related to an observation of the desired region to be observed, the report indicating that the endoscope fell short of being inserted up to the desired region to be observed; and record the handover information.

### CROSS REFERENCES TO RELATED APPLICATIONS

This application is a continuation of International Application No. PCT/JP2023/022380, filed on Jun. 16, 2023, the entire contents of which are incorporated herein by reference.

### BACKGROUND

### 1. Technical Field

The present disclosure relates to a medical assistance apparatus, a medical assistance system, and a method of operating a medical assistance apparatus that provide assistance in endoscopy.

### 2. Related Art

A technique for extracting in-body images acquired by imaging of the inside of a body, from a group of numerous images in endoscopy has been known (see, for example, Japanese Unexamined Patent Application, Publication No. 2005-124965). In this technique, the in-body images are extracted from the group of numerous images by determining whether or not images are in-body images on the basis of color component information on pixel values included in the images.

Furthermore, in a known technique, input of an internal organ is enabled when a representative image is selected from multiple images in an endoscopy management system, information is thereby input at the times images are captured or immediately after the images are captured, the information indicating the internal organs in which the images were captured, and insurance points corresponding to the number of the internal organs is thereby counted efficiently (see, for example, Japanese Unexamined Patent Application, Publication No. 2003-151483).

### SUMMARY

In some embodiments, a medical assistance apparatus includes a processor including hardware. The processor being configured to: acquire usage status information related to a usage status of an endoscope from a time point at which the endoscope is outside a body of a subject to a time point at which the endoscope observes a desired region to be observed in a body cavity of the subject; determine, based on the usage status information, whether or not the endoscope has been inserted into the body cavity or whether or not the endoscope has reached the desired region to be observed; when it is determined that the endoscope has not been inserted into the body cavity or that the endoscope has not reached the desired region to be observed, generate handover information including each condition and handover content in association with each other for a report related to an observation of the desired region to be observed, the report indicating that the endoscope fell short of being inserted up to the desired region to be observed; and record the handover information.

In some embodiments, a medical assistance system includes: an endoscope; a control apparatus configured to control the endoscope; an image diagnosis apparatus; and a medical assistance apparatus. The endoscope includes: an insertion portion configured to be inserted into a subject; and a distal end portion that is provided at a distal end of the insertion portion, the distal end portion being configured to successively generate an image data group by continuously imaging multiple regions to be observed that have been preset for the subject, and output the image data group to the control apparatus, and the image diagnosis apparatus is configured to generate detection result information obtained by detecting each of the multiple regions to be observed from the image data group by using the image data group and an inference model that has been pretrained with features of internal organs and regions, and output the detection result information to the control apparatus, the medical assistance apparatus includes a processor comprising hardware, and the processor is configured to acquire usage status information related to a usage status of the endoscope up to observation of a region to be observed predetermined for the subject, determine, based on the usage status information, whether or not the endoscope has reached the region to be observed, generate, based on a determination result on whether or not the endoscope has reached the region to be observed and on the usage status information, handover information for a report related to an observation of the region to be observed, and record the handover information.

In some embodiments, provided is a method of operating a medical assistance apparatus. The method includes: acquiring usage status information related to a usage status of an endoscope from a time point at which the endoscope is outside a body of a subject to a time point at which the endoscope observes a desired region to be observed in a body cavity of the subject; determining, based on the usage status information, whether or not the endoscope has reached the desired region to be observed; when it is determined that the endoscope has not reached the desired region to be observed based on a determination result on whether or not the endoscope has reached the desired region to be observed and on the usage status information, generating handover information including each condition and handover content in association with each other for a report related to an observation of the desired region to be observed, the report indicating that the endoscope fell short of being inserted up to the desired region to be observed; and recording the handover information.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of a medical assistance system according to an embodiment;

FIG. 2 is a block diagram illustrating a functional configuration of main parts of an endoscope and a control apparatus, according to the embodiment;

FIG. 3 is a block diagram illustrating a functional configuration of an image diagnosis apparatus, according to the embodiment;

FIG. 4 is a block diagram illustrating a functional configuration of a medical assistance apparatus, according to the embodiment;

FIG. 5 is a flowchart illustrating an outline of a process executed by the medical assistance system according to the embodiment;

FIG. 6 is a diagram illustrating an example of: an observation order for internal organs and regions; and a list of report items;

FIG. 7 is a diagram illustrating another example of: the observation order for internal organs and regions; and the list of report items;

FIG. 8 is a flowchart illustrating an outline of an endoscopic imaging process in FIG. 5;

FIG. 9 is a flowchart illustrating an outline of a report preparation process in FIG. 5;

FIG. 10 is a diagram schematically illustrating an outline of how a discrimination unit included in the medical assistance apparatus according to the embodiment discriminates an image data group into a first image data group and a second image data group;

FIG. 11 is a diagram illustrating an example of a handover information table generated by a generation unit included in the medical assistance apparatus according to the embodiment, the handover information table including each condition and handover information in association with each other; and

FIG. 12 is a flowchart illustrating an outline of a process for an examination yet to be completed in FIG. 5.

### DETAILED DESCRIPTION

An embodiment of the present disclosure will hereinafter be described in detail with reference to the drawings. The present disclosure is not to be limited by the following embodiment. The drawings referred to in the following description merely illustrate shapes, sizes, and positional relations schematically to enable the present disclosure to be understood. That is, the present disclosure is not to be limited only to the shapes, sizes, and positional relations exemplified by the drawings.

Furthermore, like reference signs will be assigned to like parts in the drawings.

### Overall Configuration of Medical Assistance System

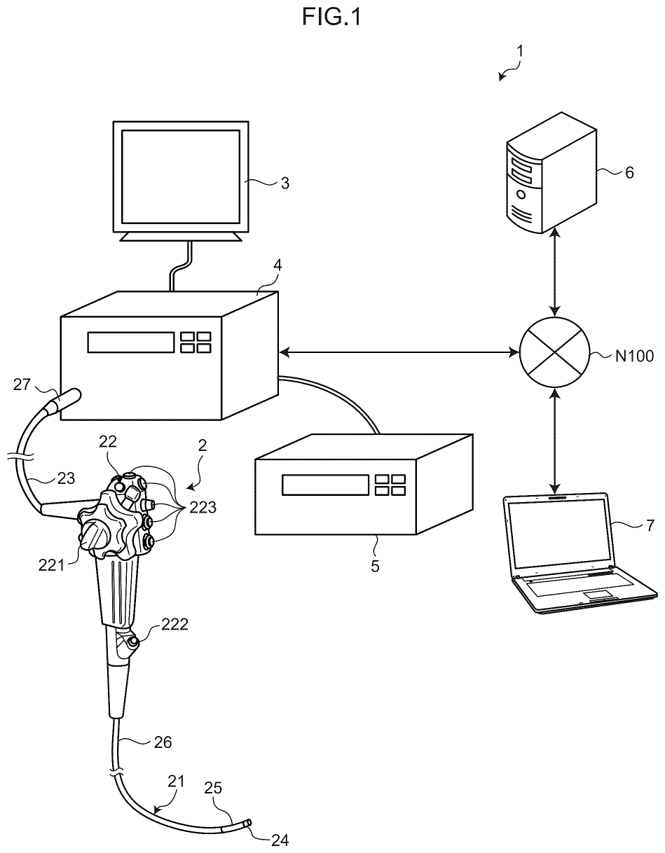

FIG. 1 is a diagram illustrating an overall configuration of a medical assistance system according to an embodiment. In a medical assistance system 1 illustrated in FIG. 1, an insertion portion 21 of an endoscope 2 is inserted from the mouth to the esophagus of a subject, such as a human or an animal, or from the anus to the large intestine of the subject, the inside of the subject is thereby continuously imaged, and an image data group thus captured is displayed as display images along a time series on a display apparatus 3.

Furthermore, in the medical assistance system 1, on the basis of the image data group input from a control apparatus 4 and an inference model (a trained model) trained beforehand using training data acquired by imaging internal organs, regions, and lesions of subjects, an image diagnosis apparatus 5 (computer aided detection: CAD) performs detection (recognition or estimation) of the internal organs and regions of multiple regions to be observed captured in image data, the insertion speed of the insertion portion 21, and any feature area including a polyp or cancer, and outputs results of this detection to the control apparatus 4. An operating surgeon, such as a medical doctor, performs observation and treatment of the subject while checking the display images displayed on the display apparatus 3 and the results of the detection (results of the estimation) from the image diagnosis apparatus 5.

Furthermore, in the medical assistance system 1, a medical assistance apparatus 6 acquires the image data group input from the control apparatus 4 via a network N100 and detection result information including the results of the detection (results of the estimation) from the image diagnosis apparatus 5 and records the acquired image data group and the detection result information in association with each other. The medical assistance system 1 then outputs various data according to a request from a laptop personal computer 7 of an operating surgeon via the network N100.

Furthermore, in the medical assistance system 1, report information on an examination report on the subject is prepared by the operating surgeon on the laptop personal computer 7 and this report information is output to and recorded in the medical assistance apparatus 6.

Thereafter, on the basis of the report information and results of diagnosis of biological cells acquired by a biopsy during observation of the subject, the operating surgeon makes a diagnosis and holds a conference for the subject.

As illustrated in FIG. 1, the medical assistance system 1 includes the endoscope 2, the display apparatus 3, the control apparatus 4, the image diagnosis apparatus 5, and the medical assistance apparatus 6.

By imaging the inside of a subject, the endoscope 2 continuously generates image data (RAW data) and successively outputs the image data to the control apparatus 4. The endoscope 2 includes, as illustrated in FIG. 1, the insertion portion 21, an operating portion 22, and a universal cord 23.

At least part of the insertion portion 21 has flexibility and the insertion portion 21 is inserted in a subject. As illustrated in FIG. 1, the insertion portion 21 includes a distal end portion 24 provided at a distal end of the insertion portion 21, a bending portion 25 that is connected to a proximal end (toward an operating portion 22) of the distal end portion 24 and is configured to be bendable, and a flexible tube portion 26 that is connected to a proximal end of the bending portion 25, has flexibility, and is elongated.

The operating portion 22 is connected to a proximal end portion of the insertion portion 21. The operating portion 22 receives various operations for the endoscope 2. This operating portion 22 has, provided therein, as illustrated in FIG. 1, a bending knob 221, an insertion opening 222, and a plurality of operating parts 223.

The bending knob 221 is configured to be rotatable according to a user operation by a user, such as an operating surgeon. By rotating, the bending knob 221 causes a bending mechanism (not illustrated in the figure) to operate, the bending mechanism being, for example, a wire that is provided in the insertion portion 21 and is made of metal or resin. The bending portion 25 is thereby bent.

The insertion opening 222 is an insertion opening in communication with a treatment tool channel (not illustrated in the figure) which is a conduit extending from the distal end of the insertion portion 21, the insertion opening being for inserting a treatment tool through the treatment tool channel from the outside of the endoscope 2.

The plurality of operating parts 223 includes buttons for receiving various operations from a user, such as an operating surgeon, and outputs operation signals according to the various operations to the control apparatus 4 via the universal cord 23. Examples of the various operations may include a release operation for instructing imaging of a still image using the endoscope 2 and an operation to switch the observation mode of the endoscope 2 to a normal light observation mode or a special observation mode.

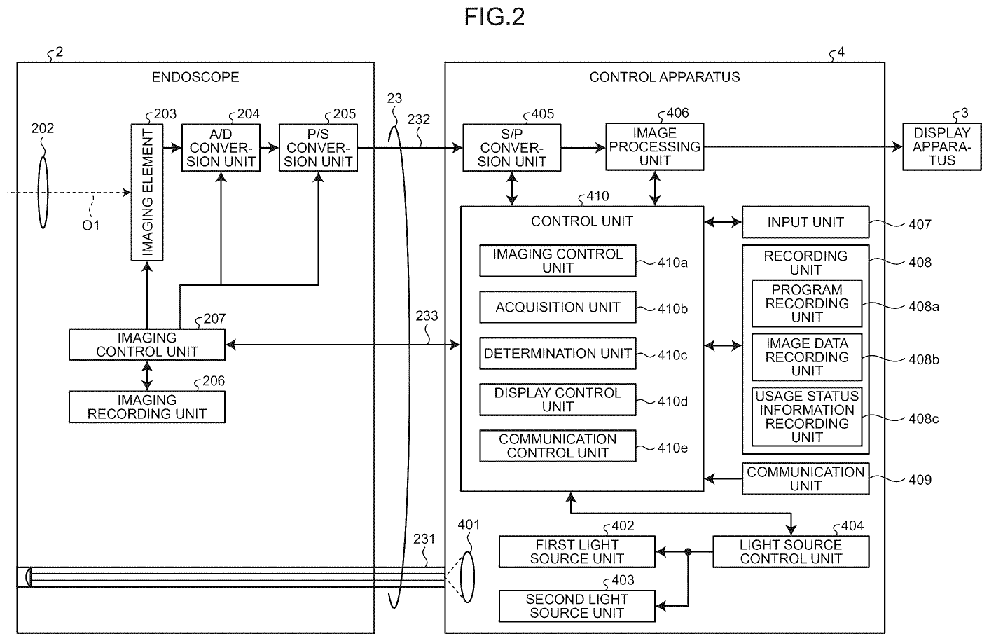

The universal cord 23 is a cord that extends from the operating portion 22 in a direction different from the direction in which the insertion portion 21 extends from the operating portion 22, the cord having, provided therein, a light guide 231 (see FIG. 2) including an optical fiber, a first signal line 232 (see FIG. 2) that transmits the image data mentioned above, and a second signal line 233 (see FIG. 2) that transmits the operation signals mentioned above. As illustrated in FIG. 1, a first connector unit 27 is provided at a proximal end of the universal cord 23. The first connector unit 27 is detachably connected to the control apparatus 4.

The display apparatus 3 includes a liquid crystal or organic electroluminescence (EL) display monitor and displays, under control by the control apparatus 4, display images based on image data that have been subjected to image processing at the control apparatus 4 and various information related to the endoscope 2.

The control apparatus 4 is implemented using a processor that is a processing device having hardware, such as a graphics processing unit (GPU), a field programmable gate array (FPGA), or a central processing unit (CPU), and a memory that is a temporary storage area used by the processor. The control apparatus 4 integrally controls operation of each unit of the endoscope 2, according to a program recorded in the memory.

The image diagnosis apparatus 5 (CAD) performs detection (estimation) of the internal organs and regions captured in image data, the insertion speed of the insertion portion 21, and any feature area (abnormal area or lesion candidate area) including a polyp or cancer by using an image data group input from the control apparatus 4 and a trained model that has been trained with training data beforehand and outputs detection result information including results of this detection and detection times in association with each other, to the control apparatus 4. The image diagnosis apparatus 5 is implemented using a processor that is a processing device having hardware, such as a GPU, an FPGA, or a CPU, and a memory that is a temporary storage area used by the processor.

The medical assistance apparatus 6 sequentially records various data input from the control apparatus 4 and recognition results from the image diagnosis apparatus 5 and transmits data according to a request from the laptop personal computer 7. The medical assistance apparatus 6 is implemented using a processor that is a processing device having hardware, such as a GPU, an FPGA, or a CPU, and a memory that is a temporary storage area used by the processor. Furthermore, the medical assistance apparatus 6 is configured using a hard disk drive (HDD) and a solid state drive (SSD).

### Functional Configuration of Main Parts of Medical Assistance System

A functional configuration of main parts of the medical assistance system 1 described above will be described next.

FIG. 2 is a block diagram illustrating a functional configuration of main parts of the endoscope 2 and the control apparatus 4. The endoscope 2 and the control apparatus 4 will be described hereinafter in this order and the image diagnosis apparatus 5 and the medical assistance apparatus 6 will be described thereafter.

### Functional Configuration of Endoscope

A configuration of the endoscope 2 will be described first.

As illustrated in FIG. 2, the endoscope 2 includes an illumination optical system 201, an imaging optical system 202, an imaging element 203, an A/D conversion unit 204, a P/S conversion unit 205, an imaging recording unit 206, and an imaging control unit 207. The illumination optical system 201, the imaging optical system 202, the imaging element 203, the A/D conversion unit 204, the P/S conversion unit 205, the imaging recording unit 206, and the imaging control unit 207 are each arranged in the distal end portion 24.

The illumination optical system 201 includes one or more lenses and emits illumination light supplied from the light guide 231 to a subject.

The imaging optical system 202 includes one or more lenses and forms a subject image on a light receiving surface of the imaging element 203 by condensing light, such as reflected light reflected from a subject, returned light from the subject, and fluorescence emitted from the subject. Furthermore, movement of the lens or lenses along an optical axis direction O1 in the imaging optical system 202 enables the focal length and the angle of view to be changed.

The imaging element 203 is configured using a charge coupled device (CCD) or complementary metal oxide semiconductor (CMOS) imaging sensor having any one of color filters forming a Bayer array (RGGB) arranged on each of a plurality of pixels arranged in a two-dimensional matrix. Under control by the imaging control unit 207, the imaging element 203 receives a subject image formed by the imaging optical system 202 and generates a captured image (analog signal) by photoelectrically converting the subject image. In this embodiment, the imaging element 203 may include an image sensor integrated with a TOF sensor that acquires subject distance information (hereinafter, referred to as depth map information) by a Time-of-Flight (TOF) method. The depth map information is information on a subject distance detected for each pixel position, the subject distance being from the position of the imaging element 203 (the position of the distal end portion 24) to a corresponding position on an observation target, the corresponding position corresponding to a pixel position on the captured image. An image sensor including a phase difference sensor may be adopted as a component that generates the depth map information, without being limited to the TOF sensor mentioned above. The depth map information and the captured image will hereinafter be collectively referred to as image data. The imaging element 203 outputs the image data to the A/D conversion unit 204.

The A/D conversion unit 204 is configured using an A/D conversion circuit, for example. Under control by the imaging control unit 207, the A/D conversion unit 204 performs A/D conversion processing of analog image data input from the imaging element 203 and outputs the converted image data to the P/S conversion unit 205.

The P/S conversion unit 205 is configured using a P/S conversion circuit, for example, and under control by the imaging control unit 207, performs parallel/serial conversion of the digital image data input from the A/D conversion unit 204 and outputs the converted image data to the control apparatus 4 via the first signal line 232.

An E/O conversion unit that converts the image data into an optical signal may be provided instead of the P/S conversion unit 205 and the image data may be output to the control apparatus 4 by means of the optical signal. Furthermore, the image data may be transmitted to the control apparatus 4 by wireless communication, such as Wireless Fidelity (Wi-Fi) (registered trademark).

The imaging recording unit 206 includes a non-volatile memory and a volatile memory and has, recorded therein, various information (for example, pixel information on the imaging element 203) related to the endoscope 2. Furthermore, the imaging recording unit 206 has, recorded therein, various setting data and control parameters transmitted from the control apparatus 4 via the second signal line 233.

The imaging control unit 207 is implemented using: a timing generator (TG); a processor that is a processing device having hardware, such as a CPU; and a memory that is a temporary storage area used by the processor. On the basis of setting data received from the control apparatus 4 via the second signal line 233, the imaging control unit 207 controls operation of each of the imaging element 203, the A/D conversion unit 204, and the P/S conversion unit 205.

### Configuration of Control Apparatus

A configuration of the control apparatus 4 will be described next.

As illustrated in FIG. 2, the control apparatus 4 includes a condenser lens 401, a first light source unit 402, a second light source unit 403, a light source control unit 404, an S/P conversion unit 405, an image processing unit 406, an input unit 407, a recording unit 408, a communication unit 409, and a control unit 410.

The condenser lens 401 condenses light emitted by each of the first light source unit 402 and the second light source unit 403 and outputs the condensed light to the light guide 231.

Under control by the light source control unit 404, the first light source unit 402 supplies, as illumination light, white light (normal light), which is visible light, to the light guide 231 by emitting the white light. This first light source unit 402 is configured using a collimator lens, a white light emitting diode (LED) lamp, and a driver, for example. The first light source unit 402 may supply white light that is visible light by simultaneous light emission using a red LED lamp, a green LED lamp, and a blue LED lamp. Furthermore, the first light source unit 402 may include a halogen lamp or a xenon lamp.

Under control by the light source control unit 404, the second light source unit 403 supplies, as illumination light, special light having a predetermined wavelength band to the light guide 231 by emitting the special light. This special light is light used in narrow band imaging (NBI) using narrow band light including 390 to 445 nm and 530 to 550 nm. The special light may of course be amber-colored light (600 nm and 630 nm) used in red dichromatic imaging (RDI), other than the narrow band light.

The light source control unit 404 is implemented using: a processor having hardware, such as an FPGA or a CPU; and a memory that is a temporary storage area used by the processor. On the basis of control data input from the control unit 410, the light source control unit 404 controls light emission timings and light emission durations of the first light source unit 402 and the second light source unit 403.

Under control by the control unit 410, the S/P conversion unit 405 performs serial/parallel conversion of image data received from the endoscope 2 via the first signal line 232 and outputs the converted image data to the image processing unit 406. In a case where the endoscope 2 outputs the image data as an optical signal, an O/E conversion unit that converts the optical signal into an electric signal may be provided instead of the S/P conversion unit 405. Furthermore, in a case where the endoscope 2 transmits the image data by wireless communication, a communication module capable of receiving a wireless signal may be provided instead of the S/P conversion unit 405.

The image processing unit 406 is implemented using: a processor having hardware, such as a GPU or an FPGA; and a memory that is a temporary storage area used by the processor. Under control by the control unit 410, the image processing unit 406 performs predetermined image processing of image data that are parallel data input from the S/P conversion unit 405 and outputs the processed image data to the display apparatus 3. Examples of the predetermined image processing may include demosaicing processing, white balance processing, gain adjustment processing, γ correction processing, and format conversion processing.

The input unit 407 is configured using any of a mouse, a foot switch, a keyboard, a button, a switch, and a touch panel, and receives a user operation from a user, such as an operating surgeon, and outputs an operation signal corresponding to the user operation, to the control unit 410.

The recording unit 408 is implemented using any of a volatile memory, a non-volatile memory, a solid state drive (SSD), a hard disk drive (HDD), and a recording medium, such as a memory card. The recording unit 408 has, recorded therein, data including various parameters required in operation of the control apparatus 4 and the endoscope 2. Furthermore, the recording unit 408 has: a program recording unit 408a that has, recorded therein, various programs for operating the endoscope 2 and the control apparatus 4; an image data recording unit 408b that has, recorded therein, image data; and a usage status information recording unit 408c that has, recorded therein, usage status information.

The image data recording unit 408b has, recorded therein, an image data group and still image data, in association with patient information, the image data group having been generated by the endoscope 2 continuously imaging multiple regions to be observed in a subject under control by the control unit 410.

The usage status information recording unit 408c has, recorded therein, usage status information. The usage status information includes detection result information input from the image diagnosis apparatus 5, detection times for these detection results, still image data, imaging times at which the still image data were captured, cleaning history information related to a cleaning history of the endoscope 2, and the patient information. The still image data are image data captured by the imaging element 203 upon imaging being instructed by a release signal input from the operating portion 22, the image data being higher in resolution than live view images successively generated by the imaging element 203.

Under control by the control unit 410, the communication unit 409 transmits various information to the medical assistance apparatus 6 via the image diagnosis apparatus 5 and the network N100, receives various information from the image diagnosis apparatus 5 and the medical assistance apparatus 6, and outputs the various information received, to the control unit 410. Specifically, under control by the control unit 410, the communication unit 409 transmits a group of temporally consecutive image data that have been subjected to image processing by the image processing unit 406, to the image diagnosis apparatus 5, receives detection results including internal organs, regions and any feature area detected at the image diagnosis apparatus 5, and transmits the detection results to the control unit 410. Furthermore, under control by the control unit 410, the communication unit 409 transmits the image data group, the still image data, and detection result information including the detection results from the image diagnosis apparatus 5, to the medical assistance apparatus 6 and receives, from the medical assistance apparatus 6, the patient information on the subject and observation information indicating an observation order of sites to be observed in an examination of the subject. The communication unit 409 is configured using a communication module, for example.

The control unit 410 corresponds to a second processor according to the present disclosure. This control unit 410 is implemented using the second processor that is a processing device having hardware, such as an FPGA or a CPU, and a memory that is a temporary storage area used by the second processor. The control unit 410 integrally controls each unit included in the endoscope 2 and the control apparatus 4. The control unit 410 has an imaging control unit 410a, an acquisition unit 410b, a determination unit 410c, a display control unit 410d, and a communication control unit 410e.

The imaging control unit 410a controls imaging implemented by the endoscope 2. The imaging control unit 410a generates a group of temporally consecutive image data by causing the endoscope 2 to implement imaging continuously. Furthermore, in a case where a release signal has been input from the operating portion 22, the imaging control unit 410a causes the imaging element 203 to capture still image data.

The acquisition unit 410b acquires image data from the endoscope 2 via the S/P conversion unit 405 or the image processing unit 406 and records the image data acquired into the image data recording unit 408b. Furthermore, the acquisition unit 410b acquires detection result information from the image diagnosis apparatus 5 via the communication unit 409 and records the detection result information into the usage status information recording unit 408c.

On the basis of observation order information on a final region to be reached set by an observation order for report preparation and a region in the subject detected by the image diagnosis apparatus 5, the determination unit 410c makes a determination on whether or not the distal end portion 24 of the endoscope 2 has reached the final region to be reached, and records a result of this determination as a piece of usage status information in the usage status information recording unit 408c. Furthermore, the determination unit 410c makes a determination on whether or not the final region to be reached is yet to be imaged and records a result of this determination as a piece of usage status information in the usage status information recording unit 408c. Specifically, the determination unit 410c determines whether or not still image data have been recorded in the image data recording unit 408b, the still image data having been acquired by imaging of the final region to be reached of the internal organs and regions set by the observation order (insertion order) for report items in the usage status information recording unit 408c.

In a case where the determination unit 410c determines that the distal end portion 24 of the endoscope 2 has reached the final region to be reached, the display control unit 410d causes the display apparatus 3 to display information indicating that the distal end portion 24 of the endoscope 2 has reached the final region to be reached in the subject.

Via the network N100, the communication control unit 410e causes the communication unit 409 to transmit various information to the medical assistance apparatus 6 and causes the communication unit 409 to receive various information from the medical assistance apparatus 6.

### Functional Configuration of Image Diagnosis Apparatus

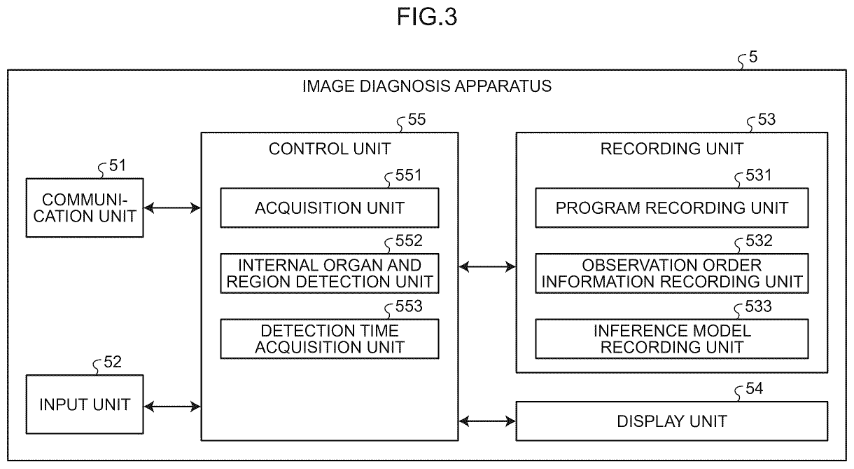

A functional configuration of the image diagnosis apparatus 5 will be described next. FIG. 3 is a block diagram illustrating the functional configuration of the image diagnosis apparatus 5. The image diagnosis apparatus 5 illustrated in FIG. 3 includes a communication unit 51, an input unit 52, a recording unit 53, a display unit 54, and a control unit 55.

Under control by the control unit 55, the communication unit 51 receives an image data group generated by the endoscope 2 and internal organ and region order information indicating an observation order of internal organs and regions in a subject who is a patient, from the control apparatus 4 via the network N100 and outputs the received image data group and internal organ and region order information to the control unit 55. Furthermore, under control by the control unit 55, the communication unit 51 outputs detection result information including detection results of detection of internal organs of the subject and detection times at which these detection results were detected that are input from the control unit 55 via the network N100, to the control apparatus 4. The communication unit 51 is configured using a communication module, for example.

The input unit 52 is configured using any of a button, a switch, and a touch panel, for example, and receives various input and outputs the various input received, to the control unit 55.

The recording unit 53 is configured using any of a volatile memory, a non-volatile memory, an SSD, an HDD, and a recording medium, such as a memory card. The recording unit 53 has, recorded therein, data including various parameters required in operation of the image diagnosis apparatus 5. Furthermore, the recording unit 53 has a program recording unit 531 having, recorded therein, various programs for operating the image diagnosis apparatus 5, an observation order recording unit 532, and an inference model recording unit 533.

The observation order recording unit 532 has, recorded therein, observation order information having, set therein, an observation order for multiple preset sites to be observed in internal organs and regions for observation of a subject using the endoscope 2, the observation order information having been input from the medical assistance apparatus 6 described later.

The inference model recording unit 533 has, recorded therein, an inference model used for detection of internal organs, regions, and any feature area on the basis of image data. The inference model is a model to which image data from the endoscope 2 are input and from which internal organs, regions, and any feature area included in the image data are output. This inference model is, for example, a model generated by machine learning using artificial intelligence (AI). Specifically, the inference model is, for example, a deep learning model that is trained using training data having internal organs, regions, and feature areas, such as lesions, of subjects assigned to image data acquired by imaging of the subjects, and that outputs internal organs, regions, and any feature area included in image data.

Under control by the control unit 55, the display unit 54 displays various information related to the image diagnosis apparatus 5. The display unit 54 is configured using a liquid crystal display or an organic EL display, for example.

The control unit 55 is implemented using: a processor having hardware, such as an FPGA or a CPU; and a memory that is a temporary storage area used by the processor. The control unit 55 integrally controls each unit included in the medical assistance apparatus 6. The control unit 55 has an acquisition unit 551, an internal organ and region detection unit 552, and a detection time acquisition unit 553.

The acquisition unit 551 acquires, along a time-series, image data successively generated by the endoscope 2, from the control apparatus 4 via the communication unit 51.

On the basis of the image data successively acquired by the acquisition unit 551 and the inference model recorded in the inference model recording unit 533, the internal organ and region detection unit 552 detects internal organs, regions, and any feature area of the subject included in the image data.

The detection time acquisition unit 553 acquires detection times at which the internal organ and region detection unit 552 detected the internal organs, regions, and any feature area of the subject included in the image data. For example, the detection time acquisition unit 553 acquires, as the detection times, times measured by a TG not illustrated in the figure when the internal organ and region detection unit 552 detects the internal organs, regions, and any feature included the image data.

### Functional Configuration of Medical Assistance Apparatus

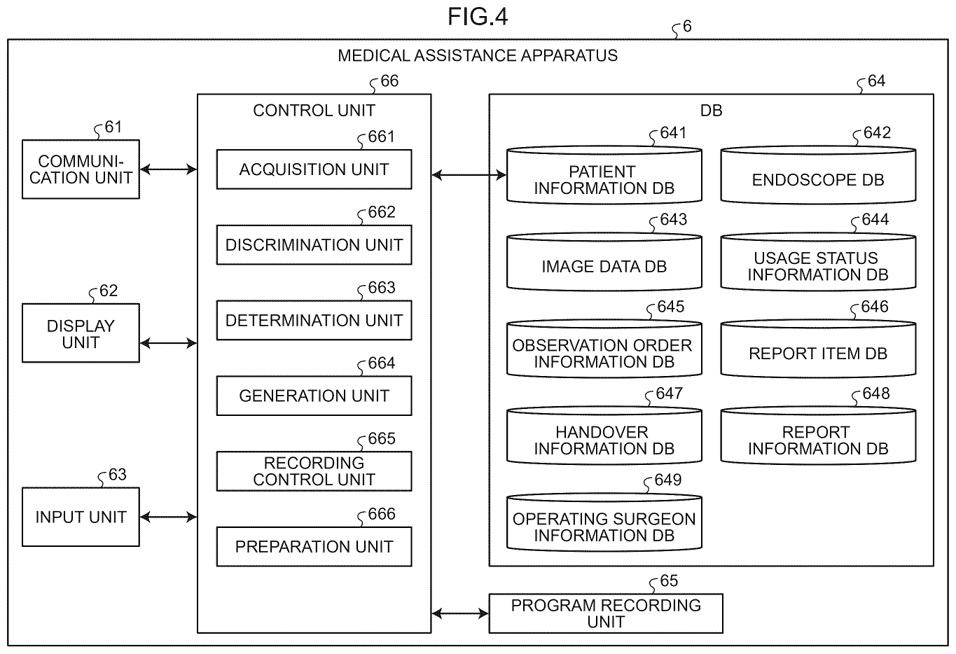

A functional configuration of the medical assistance apparatus 6 will be described next. FIG. 4 is a block diagram illustrating the functional configuration of the medical assistance apparatus 6. The medical assistance apparatus 6 illustrated in FIG. 4 includes a communication unit 61, a display unit 62, an input unit 63, a database 64 (hereinafter, simply referred to as the “DB 64”), a program recording unit 65, and a control unit 66.

Under control by the control unit 66, the communication unit 61 transmits various information to the control apparatus 4 and the image diagnosis apparatus 5 and receives various information from the control apparatus 4 and the image diagnosis apparatus 5, via the network N100. The communication unit 61 is configured using a communication module, for example.

Under control by the control unit 66, the display unit 62 displays various information related to the medical assistance apparatus 6. The display unit 62 is configured using a liquid crystal display or an organic EL display, for example.

The input unit 63 receives various input according to operations from the outside and outputs the various input received, to the control unit 66. The input unit 63 is configured using any of a button, a keyboard, a switch, and a touch panel, for example.

The DB 64 has, recorded therein, various information. The DB 64 is configured using an HDD and an SSD, for example. The DB 64 has a patient information DB 641, an endoscope DB 642, an image data DB 643, a usage status information DB 644, an observation order information DB 645, a report item DB 646, a handover information DB 647, a report information DB 648, and an operating surgeon information DB 649.

The patient information DB 641 has, recorded therein, for each patient, a patient ID identifying the patient, the patient's date of birth, a doctor in charge of the patient, the patient's address, and allergy information on the patient.

The endoscope DB 642 has, recorded therein, for each endoscope 2, the type of the endoscope 2, the endoscope ID, the repair history of the endoscope 2, the maintenance status of the endoscope 2, cleaning history information related to the cleaning history of the endoscope 2, and the usage history of and intended use information on the endoscope 2.

The image data DB 643 has, recorded therein, each patient ID or each operating surgeon ID, associated with an image data group captured by the endoscope 2.

The usage status information DB 644 has, recorded therein, usage status information related to usage statuses of the endoscope 2 up to observation of a predetermined region to be observed in the subject. The usage status information includes: insertion information indicating whether or not the endoscope 2 was inserted in the body of the subject, the insertion information being based on the image data group captured by the endoscope 2; operation information on an operating surgeon for the operating portion 22 included in the endoscope 2; cleaning history information related to the cleaning history of the endoscope 2; detection result information on detection of multiple internal organs and regions in the subject by the image diagnosis apparatus 5, the detection being based on the image data group captured by the endoscope 2; feature area detection information indicating whether or not a feature area in the body of the subject has been detected by the image diagnosis apparatus 5 on the basis of the image data group captured by the endoscope 2; and release information related to a history of a release signal from the operating parts 223 included in the endoscope 2.

The observation order information DB 645 has, recorded therein, an observation order of internal organs and regions for the endoscope 2 set for each institution or operating surgeon ID.

The report item DB 646 has, recorded therein, report items for the endoscope 2 set for each institution or operating surgeon ID.

The handover information DB 647 has, recorded therein, handover information to be handed over in later described report information on a subject generated by the medical assistance apparatus 6.

The report information DB 648 has, recorded therein, report information for the endoscope 2 prepared by the operating surgeon (image interpreter) and report information automatically generated by the medical assistance apparatus 6, both in association with each patient ID or operating surgeon ID.

The operating surgeon information DB 649 has, recorded therein, the full name, title, affiliation, and contact information, in association with each operating surgeon ID.

The program recording unit 65 is configured using a volatile memory, a non-volatile memory, and an SSD, for example, and has, recorded therein, various programs for operating the medical assistance apparatus 6.

The control unit 66 corresponds to a first processor according to the present disclosure. This control unit 66 is implemented using the first processor having hardware, such as an FPGA or a CPU, and a memory that is a temporary storage area used by the first processor. The control unit 66 integrally controls each unit included in the medical assistance apparatus 6. The control unit 66 has an acquisition unit 661, a discrimination unit 662, a determination unit 663, a generation unit 664, a recording control unit 665, and a preparation unit 666.

The acquisition unit 661 acquires an image data group and usage status information from the control apparatus 4 via the network N100.

On the basis of the image data group and the usage status information acquired by the acquisition unit 661, the discrimination unit 662 discriminates between a first image data group captured upon observation of a predetermined region to be observed and a second image data group captured after the observation of the predetermined region to be observed.

On the basis of the usage status information acquired by the acquisition unit 661, the determination unit 663 determines whether or not the endoscope 2 has reached the region to be observed. Specifically, on the basis of the first image data group discriminated by the discrimination unit 662 and the usage status information, the determination unit 663 determines whether or not the distal end portion 24 of the endoscope 2 has been inserted in the body of the subject. Furthermore, on the basis of insertion information included in the usage status information, the determination unit 663 determines whether or not the endoscope 2 has been inserted in the body of the subject. Furthermore, on the basis of the insertion information and detection result information included in the usage status information, the determination unit 663 makes a determination on whether or not the endoscope 2 has reached a preset region to be observed (final region to be reached). In a case where the determination unit 663 determines that the endoscope 2 has reached the region to be observed, the determination unit 663 determines, on the basis of feature area detection information included in the usage status information, whether or not any feature area has been detected in the body of the subject. Furthermore, on the basis of release information included in the usage status information, the determination unit 663 makes a determination on whether or not a still image is present.

On the basis of a result of the determination made by the determination unit 663, the determination being on whether or not the endoscope 2 has reached the region to be observed, and the usage status information, the generation unit 664 generates handover information for a report related to observation of the region to be observed for the subject. Details of the handover information generated by the generation unit 664 will be described later.

The recording control unit 665 records the handover information generated by the generation unit 664 into the handover information DB 647. In this case, the recording control unit 665 records the handover information in association with patient information or operating surgeon information, into the handover information DB 647.

The preparation unit 666 prepares report information on a subject on the basis of operation information from the input unit 63 or laptop personal computer 7, an image data group and usage status information acquired by the acquisition unit 661, patient information, and related information generated by the generation unit 664, and records this report information in association with patient information (patient ID) or operating surgeon information (operating surgeon ID), into the report information DB 648.

### Process in Medical Assistance System

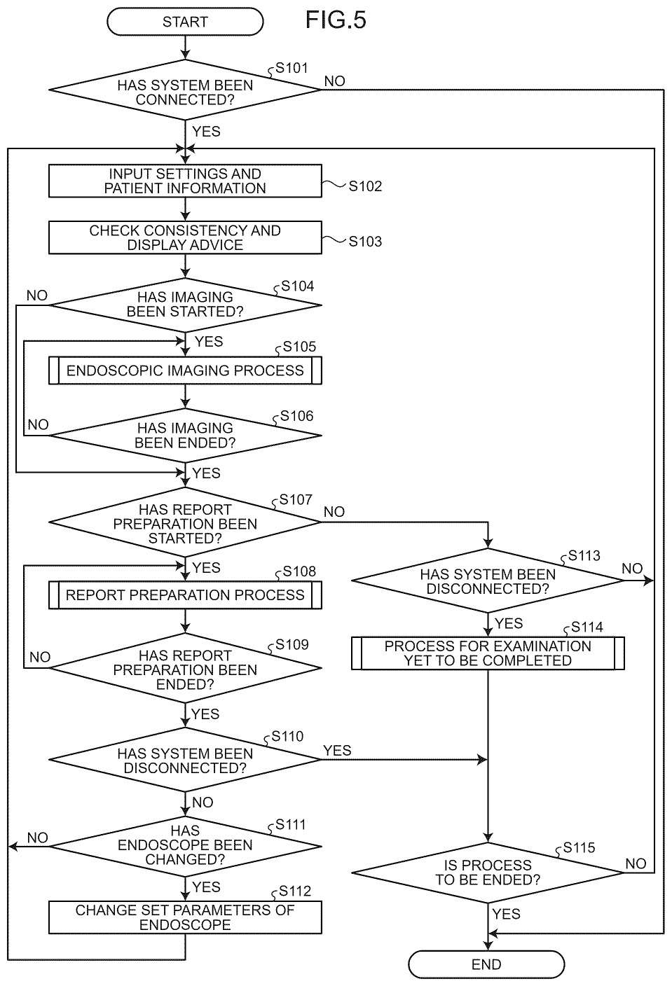

An outline of a process executed by the medical assistance system 1 will be described next. FIG. 5 is a flowchart illustrating the outline of the process executed by the medical assistance system 1.

As illustrated in FIG. 5, firstly, the control unit 66 determines whether or not the endoscope 2 has been connected to the medical assistance system 1 by the endoscope 2 being connected to the control apparatus 4 via the network N100 (Step S101). Specifically, the control unit 66 determines whether or not an endoscope ID identifying the endoscope 2 input from the control apparatus 4 via the network N100 has been input, and determines that the endoscope 2 has been connected to the medical assistance system 1 in a case where the endoscope ID identifying the endoscope 2 has been input from the control apparatus 4.

In a case where the control unit 66 determines that the endoscope 2 has been connected to the medical assistance system 1 by the endoscope 2 being connected to the control apparatus 4 (Step S101: Yes), the medical assistance system 1 proceeds to Step S102. On the contrary, in a case where the control unit 66 determines that the endoscope 2 has not been connected to the control apparatus 4 and the endoscope 2 has not been connected to the medical assistance system 1 (Step S101: No), the medical assistance system 1 ends this process.

Subsequently, the control unit 66 receives a setting of an observation order of internal organs and regions of a subject input from the laptop personal computer 7 or control apparatus 4 and records the setting into the observation order information DB 645 and receives input of a patient name and records the patient name into the patient information DB 641 (Step S102). Specifically, on the basis of an operation input from the laptop personal computer 7 of an operating surgeon, the control unit 66 receives input of patient information and records the patient information received, into the patient information DB 641, the patient information including: various information, such as the name of the patient (subject), the patient ID (subject ID) of the subject, and the patient's (subject's) date of birth; the type of the endoscope 2 used in the examination of the patient (subject); the operating surgeon ID identifying the doctor in charge; the types of report items; the content of the report items; examination appointment information on the patient (examination time and date); a pre-examination consent form; medical interview results; an informed consent form; and advance registration. Furthermore, on the basis of an operation input from the laptop personal computer 7 of the operating surgeon, the control unit 66 records observation order information related to the observation order for observation of internal organs and regions using the endoscope 2, into the observation order information DB 645.

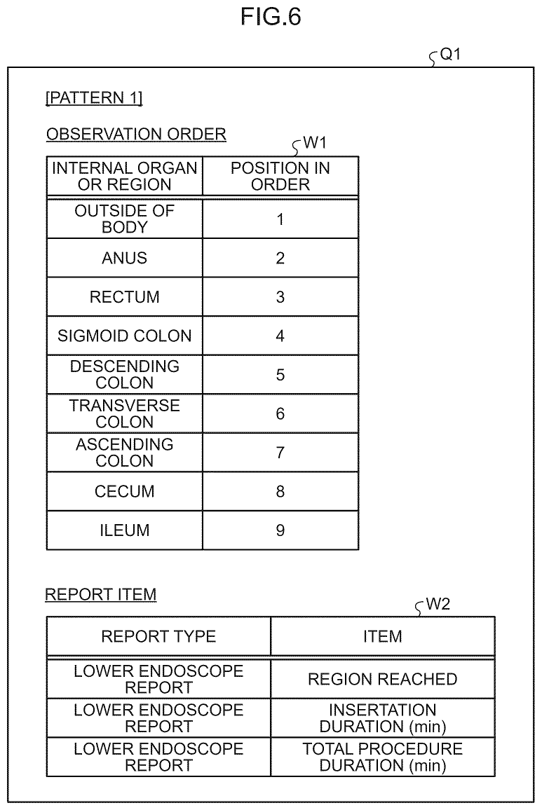

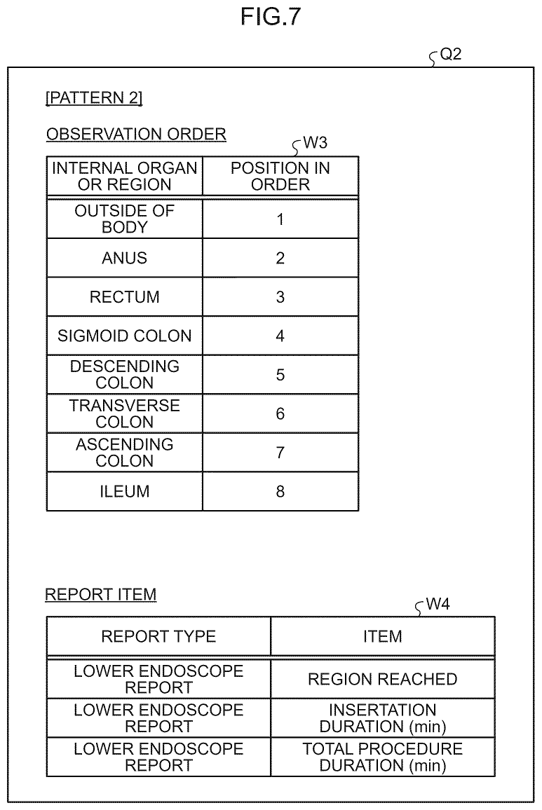

FIG. 6 is a diagram illustrating an example of: an observation order of internal organs and regions; and a list of report items. FIG. 7 is a diagram illustrating another example of: the observation order of internal organs and regions; and the list of report items.

In observation order tables W1 and W3 for internal organs and regions for a pattern Q1 illustrated in FIG. 6 and a pattern Q2 illustrated in FIG. 7, the order of observation of internal organs and regions has been set for each operating surgeon (each medical doctor) or each institution. This observation uses the endoscope 2, and the distal end portion 24 of the insertion portion 21 included in the endoscope 2 (hereinafter, simply referred to as the “distal end portion 24”) is inserted from the outside to the inside of the body of the subject. For example, the position in the order, “9”, has been set for the internal organ or region, “ileum”, in the observation order table W1 for internal organs and regions.

Similarly, in report item tables W2 and W4, report types have been set in association with items. For example, in the report item table W2, the item, “region reached”, has been set for the report type, “lower endoscope report”. A region reached herein refers to an internal organ or region at a site to be observed that the distal end portion 24 was able to reach in the subject. A case in which the pattern Q1 illustrated in FIG. 6 has been set will be described hereinafter.

By reference back to FIG. 5, description of the process from Step S103 will now be resumed.

At Step S103, on the basis of the patient information input at Step S102, past report information recorded in the report information DB 648 and associated with the patient information, and endoscope information recorded in the endoscope DB 642, the control unit 66 checks consistency between the patient who is a subject and the endoscope 2 and causes the display apparatus 3 or the laptop personal computer 7 to display advice, via the network N100. Specifically, on the basis of the patient information, the past report information recorded in the report information DB 648 and associated with the patient information, and the endoscope information recorded in the endoscope DB 642, the control unit 66 checks consistency of settings of the endoscope 2 used in observation of the patient. For example, the control unit 66 makes a consistency check determination on whether or not the endoscope 2 is of the type suitable for observation corresponding to: the observation order for the observation of the internal organs and regions set for the endoscope 2 used in the observation of the patient; the types of the report items; and the examination appointment information. In this case, if the control unit 66 determines that the endoscope 2 is of the type not suitable for the observation of the patient or the settings are not suitable, the control unit 66 displays a warning on the display apparatus 3 or the laptop personal computer 7. Furthermore, on the basis of the usage status information recorded in the usage status information recording unit 408c, the endoscope information recorded in the endoscope DB 642, and the handover information recorded in the handover information DB 647, the control unit 66 determines whether or not the endoscope 2 is yet to be cleaned, and in a case where the endoscope 2 is yet to be cleaned, the control unit 66 displays a warning on the display apparatus 3 or the laptop personal computer 7. The operating surgeon is thereby able to recognize that the endoscope 2 connected to the control apparatus 4 is not suitable for the observation of the patient.

Furthermore, on the basis of patient information, report information including past handover information recorded in the report information DB 648 associated with the patient information, and endoscope information recorded in the endoscope DB 642, the control unit 66 causes the display apparatus 3 or the laptop personal computer 7 to display advice that is handover information on: whether or not the patient presently has any allergy; the operating surgeon's findings in the previous observation of the patient; and any tumor in the patient in the previous observation and any region set in the previous observation for follow-up observation. The operating surgeon is thereby able to get a grasp of handover information for the patient from the previous operating surgeon or report author.

Subsequently, via the network N100, the control unit 66 determines whether or not imaging of the subject using the endoscope 2 has started, from the control apparatus 4 or the endoscope 2 (Step S104). Specifically, in a case where the control unit 66 receives image data or an activation signal indicating that the endoscope 2 has been activated, from the control apparatus 4 via the network N100, the control unit 66 determines that imaging of the subject using the endoscope 2 has started.

In a case where the control unit 66 determines that imaging of the subject using the endoscope 2 has started (Step S104: Yes), the medical assistance system 1 proceeds to Step S105 described later. On the contrary, in a case where the control unit 66 determines that imaging of the subject using the endoscope 2 has not started (Step S104: No), the medical assistance system 1 proceeds to Step S107 described later.

At Step S105, the medical assistance system 1 executes an endoscopic imaging process for observation by insertion of the endoscope 2 in the subject. Details of the endoscopic imaging process will be described later. After Step S105, the medical assistance system 1 proceeds to Step S106.

Subsequently, via the network N100, the control unit 66 determines whether or not imaging of the subject using the endoscope 2 has ended, from the control apparatus 4 or the endoscope 2 (Step S106). Specifically, in a case where reception of image data has stopped or a deactivation signal indicating that the endoscope 2 has been deactivated is received from the control apparatus 4 via the network N100, the control unit 66 determines that the imaging of the subject using the endoscope 2 has stopped. In a case where the control unit 66 determines that the imaging of the subject using the endoscope 2 has stopped (Step S106: Yes), the medical assistance system 1 proceeds to Step S107 described later. On the contrary, in a case where the control unit 66 determines that the imaging of the subject using the endoscope 2 has not stopped (Step S106: No), the medical assistance system 1 returns to Step S105.

At Step S107, via the network N100, the control unit 66 determines whether or not a medical doctor or an image interpretation report author has started preparing a report using the laptop personal computer 7. Specifically, the control unit 66 determines whether or not a request signal has been received from the laptop personal computer 7, the request signal requesting predetermined patient information, the image data group captured by the endoscope 2 and associated with the patient information, still image data, and detection result information including detection results from the image diagnosis apparatus 5. In a case where the control unit 66 determines that the report author has started preparing a report using the laptop personal computer 7 (Step S107: Yes), the medical assistance system 1 proceeds to Step S108 described later. On the contrary, in a case where the control unit 66 determines that the report author has not started preparing a report using the laptop personal computer 7 (Step S107: No), the medical assistance system 1 proceeds to Step S113.

At Step S108, the medical assistance system 1 executes a report preparation process of preparing a report on the basis of observation results for the subject. Details of the report preparation process will be described later.

Subsequently, via the network N100, the control unit 66 determines whether or not the report author who prepares the report, such as a medical doctor who performs image interpretation, has finished preparing the report using the laptop personal computer 7 (Step S109). Specifically, the control unit 66 determines whether or not an end signal to end report preparation has been received from the laptop personal computer 7, and in a case where the end signal has been received, the control unit 66 determines that the report preparation has ended and in a case where the end signal has not been received, the control unit 66 determines that the report preparation has not ended. In a case where the control unit 66 determines that the report author who prepares the report, such as the medical doctor who performs the image interpretation, has finished preparing the report using the laptop personal computer 7 (Step S109: Yes), the medical assistance system 1 proceeds to Step S110. On the contrary, in a case where the control unit 66 determines that the report author who prepares the report, such as the medical doctor who performs the image interpretation, has not finished preparing the report using the laptop personal computer 7 (Step S109: No), the medical assistance system 1 returns to Step S108.

At Step S110, via the network N100, the control unit 66 determines whether or not the laptop personal computer 7 or the control apparatus 4 has been disconnected from the medical assistance apparatus 6. In a case where the control unit 66 determines that the laptop personal computer 7 or the control apparatus 4 has been disconnected from the medical assistance apparatus 6 (Step S110: Yes), the medical assistance system 1 proceeds to Step S115 described later. On the contrary, in a case where the control unit 66 determines that the laptop personal computer 7 or the control apparatus 4 has not been disconnected from the medical assistance apparatus 6 (Step S110: No), the medical assistance system 1 proceeds to Step S111 described later.

At Step S111, via the network N100, the control unit 66 determines, on the basis of the endoscope ID identifying the endoscope 2 input from the control apparatus 4, whether or not the endoscope 2 connected to the control apparatus 4 has been changed. In a case where the control unit 66 determines that the endoscope 2 connected to the control apparatus 4 has been changed (Step S111: Yes), the medical assistance system 1 proceeds to Step S112 described later. On the contrary, in a case where the control unit 66 determines that the endoscope 2 connected to the control apparatus 4 has not been changed (Step S111: No), the medical assistance system 1 returns to Step S102.

At Step S112, on the basis of the endoscope ID of the endoscope 2 connected, the control apparatus 4 changes set parameters of the endoscope 2. Specifically, on the basis of the endoscope ID of the endoscope 2 connected, the control apparatus 4 changes parameters for image processing, for example, parameter for white balance. After Step S112, the medical assistance system 1 returns to Step S102.

At Step S113, via the network N100, the control unit 66 determines whether or not the laptop personal computer 7 or the control apparatus 4 has been disconnected from the medical assistance apparatus 6. In a case where the control unit 66 determines that the laptop personal computer 7 or the control apparatus 4 has been disconnected from the medical assistance apparatus 6 (Step S113: Yes), the medical assistance system 1 proceeds to Step S114 described later. On the contrary, in a case where the control unit 66 determines that the laptop personal computer 7 or the control apparatus 4 has not been disconnected from the medical assistance apparatus 6 (Step S113: No), the medical assistance system 1 returns to Step S102 described later.

At Step S114, the medical assistance system 1 executes a process for an examination yet to be completed in a case where the endoscope 2 has not been inserted in the subject yet. Details of the process for an examination yet to be completed will be described later. After Step S114, the medical assistance system 1 proceeds to Step S115.

Subsequently, the control unit 66 determines whether or not the observation of the subject using the endoscope 2 or the report preparation using the laptop personal computer 7 is to be ended (Step S115). In a case where the control unit 66 determines that the observation of the subject using the endoscope 2 or the report preparation using the laptop personal computer 7 is to be ended (Step S115: Yes), the medical assistance system 1 ends the process. On the contrary, in a case where the control unit 66 determines that the observation of the subject using the endoscope 2 or the report preparation using the laptop personal computer 7 is not to be ended (Step S115: No), the medical assistance system 1 returns to Step S102.

### Outline of Endoscopic Imaging Process

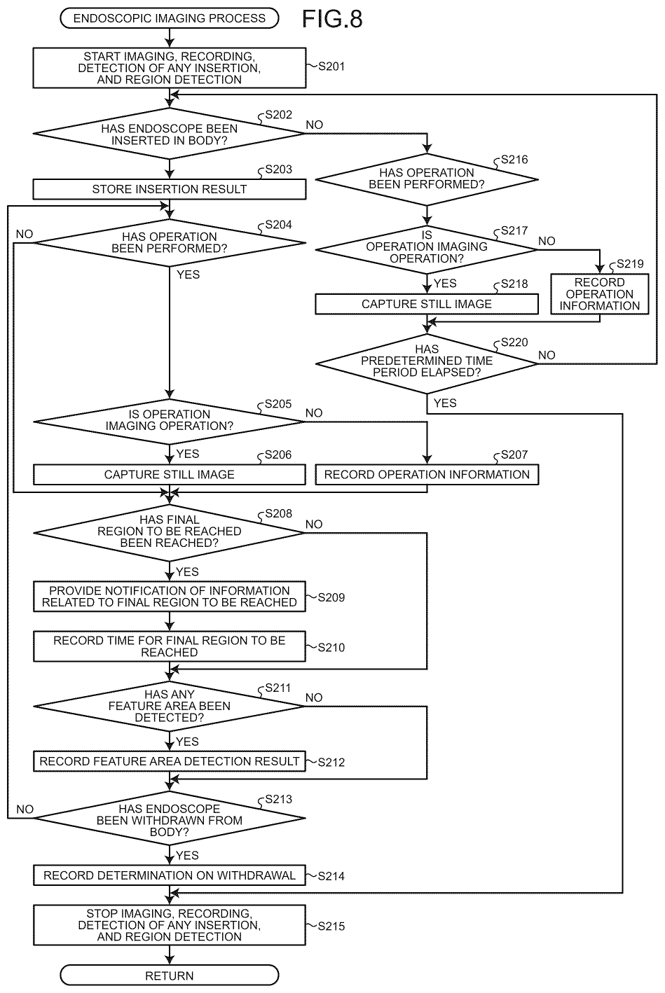

Details of the endoscopic imaging process at Step S105 in FIG. 5 will be described next. FIG. 8 is a flowchart illustrating an outline of the endoscopic imaging process.

As illustrated in FIG. 8, firstly, the control unit 410 starts: imaging by means of the endoscope 2; recording image data captured by the endoscope 2; detection of whether or not insertion into the subject has been detected by the image diagnosis apparatus 5; and region detection for internal organs and regions by means of the image diagnosis apparatus 5 (Step S201). Specifically, the imaging control unit 410a causes the endoscope 2 to start imaging. In this case, the communication control unit 410e outputs, along a time series, image data (live view images) generated by successive imaging implemented by the endoscope 2, to the image diagnosis apparatus 5. On the basis of the image data input from the control apparatus 4 and the inference model, the image diagnosis apparatus 5 then starts detecting internal organs and regions included in the image data, whether or not the distal end portion 24 of the endoscope 2 has been inserted, and any feature area, and thereby outputs detection result information to the control apparatus 4. In this case, the acquisition unit 410b starts: generating usage status information including, in association with one another, the detection result information input from the image diagnosis apparatus 5, the endoscope ID of the endoscope 2, and patient information input from the medical assistance apparatus 6 or the input unit 407; and recording the usage status information into the usage status information recording unit 408c.

On the basis of a detection result from the image diagnosis apparatus 5 and acquired by the acquisition unit 410b, the determination unit 410c determines whether or not the distal end portion 24 of the endoscope 2 has been inserted in the body of the subject (Step S202). Specifically, in a case where the anus or oral cavity is included in the detection result from the image diagnosis apparatus 5, the detection result having been acquired by the acquisition unit 410b, the determination unit 410c determines that the distal end portion 24 of the endoscope 2 has been inserted in the body of the subject. In a case where the determination unit 410c determines that the distal end portion 24 of the endoscope 2 has been inserted in the body of the subject (Step S202: Yes), the control apparatus 4 proceeds to Step S203 described later. On the contrary, in a case where the determination unit 410c determines that the distal end portion 24 of the endoscope 2 has not been inserted in the body of the subject (Step S202: No), the control apparatus 4 proceeds to Step S216 described later.

At Step S203, the acquisition unit 410b records an insertion result indicating that the distal end portion 24 of the endoscope 2 has been inserted in the body of the subject, into the usage status information recording unit 408c, the insertion result having been detected by the image diagnosis apparatus 5.

Subsequently, the determination unit 410c determines whether or not an operating surgeon has performed an operation on the operating portion 22 (Step S204). In a case where the determination unit 410c determines that the operating surgeon has performed an operation on the operating portion 22 (Step S204: Yes), the control apparatus 4 proceeds to Step S205 described later. On the contrary, in a case where the determination unit 410c determines that the operating surgeon has not performed an operation on the operating portion 22 (Step S204: No), the control apparatus 4 proceeds to Step S208 described later.

At Step S205, the determination unit 410c determines whether or not the operation performed by the operating surgeon was an imaging operation. Specifically, in a case where a release signal has been input from the operating portion 22, the determination unit 410c determines that the operating surgeon has performed the imaging operation on the operating portion 22. In a case where the control unit 410 determines that the operating surgeon has performed the imaging operation on the operating portion 22 (Step S205: Yes), the control apparatus 4 proceeds to Step S206 described later. On the contrary, in a case where the determination unit 410c determines that the operating surgeon has not performed the imaging operation on the operating portion 22 (Step S205: No), the control apparatus 4 proceeds to Step S207 described later.

At Step S206, the imaging control unit 410a causes the imaging element 203 to capture a still image (still image data). Specifically, the imaging control unit 410a records a still image file into the image data recording unit 408b, the still image file including, in association with each other, the still image data generated by the imaging element 203 and the imaging time at which the still image data were captured by the imaging element 203. The communication control unit 410e then outputs the still image file to the medical assistance apparatus 6 via the network N100. The still image data correspond to an image higher in resolution (having a larger number of pixels) than live view image data (for example, a 4K image). After Step S206, the control apparatus 4 proceeds to Step S208.

At Step S207, the acquisition unit 410b acquires operation information related to the operation performed on the operating portion 22 by the operating surgeon and records the operation information into the usage status information recording unit 408c. The operation may be a water feeding operation for the endoscope 2 or a change operation for changing the angle of view of the imaging optical system 202. The operation may of course be an operation for treatment using an energy device not illustrated in the figure. After Step S207, the control apparatus 4 proceeds to Step S208.

Subsequently, on the basis of observation order information on a final region to be reached set in the observation order for report preparation (for example, see the pattern Q1 in FIG. 6) and regions in the subject detected by the image diagnosis apparatus 5, the determination unit 410c determines whether or not the distal end portion 24 of the endoscope 2 has reached the final region to be reached (Step S208). Specifically, in a case where the final region to be reached set in the observation order is the ileum, the determination unit 410c determines that the distal end portion 24 of the endoscope 2 has reached the final region to be reached when the determination unit 410c detects that the region detected by the image diagnosis apparatus 5 is the ileum, and the determination unit 410c determines that the distal end portion 24 of the endoscope 2 has not reached the final region to be reached when the region detected by the image diagnosis apparatus 5 is not the ileum. In a case where the determination unit 410c determines that the distal end portion 24 of the endoscope 2 has reached the final region to be reached (Step S208: Yes), the control apparatus 4 proceeds to Step S209 described later. On the contrary, in a case where the determination unit 410c determines that the distal end portion 24 of the endoscope 2 has not reached the final region to be reached (Step S208: No), the control apparatus 4 proceeds to Step S211 described later.

At Step S209, the display control unit 410d causes the display apparatus 3 to display information indicating that the distal end portion 24 of the endoscope 2 has reached the final region to be reached in the subject. For example, by causing the display apparatus 3 to display information indicating that the distal end portion 24 of the endoscope 2 has reached the “ileum” set as the final region to be reached, the display control unit 410d notifies the operating surgeon of the information. The operating surgeon is thereby able to know that the distal end portion 24 of the endoscope 2 has reached the final region to be reached set in the observation order.

Subsequently, the acquisition unit 410b acquires detection result information on internal organs and regions recognized by the image diagnosis apparatus 5 and the detection time at which the distal end portion 24 of the endoscope 2 reached the final region to be reached and records the acquired detection result information and detection time into the usage status information recording unit 408c (Step S210). In this case, the acquisition unit 410b records the detection result for the detection of the final region to be reached and its detection time in association with each other, into the usage status information recording unit 408c.

Thereafter, on the basis of the detection result detected by the image diagnosis apparatus 5, the determination unit 410c determines whether or not the image diagnosis apparatus 5 has detected any feature area from the image data group generated by the endoscope 2 (Step S211). In a case where the determination unit 410c determines that the image diagnosis apparatus 5 has detected any feature area from the image data group generated by the endoscope 2 (Step S211: Yes), the control apparatus 4 proceeds to Step S212 described later. On the contrary, in a case where the determination unit 410c determines that the image diagnosis apparatus 5 has not detected any feature area from the image data group generated by the endoscope 2 (Step S211: No), the control apparatus 4 proceeds to Step S213 described later.

At Step S212, the acquisition unit 410b records any feature area detected by the image diagnosis apparatus 5 and the detection time at which the image diagnosis apparatus 5 detected the feature area, into the usage status information recording unit 408c.

Subsequently, on the basis of a detection result from the image diagnosis apparatus 5, the determination unit 410c determines whether or not the distal end portion 24 of the endoscope 2 has been withdrawn from the inside of the body of the subject. In a case where the determination unit 410c determines that the distal end portion 24 of the endoscope 2 has been withdrawn from the inside of the body of the subject (Step S213: Yes), the control apparatus 4 proceeds to Step S214 described later. On the contrary, in a case where the determination unit 410c determines that the distal end portion 24 of the endoscope 2 has not been withdrawn from the inside of the body of the subject (Step S213: No), the control apparatus 4 proceeds to Step S204 described later.

At Step S214, on the basis of a detection result from the image diagnosis apparatus 5, the control unit 410 records usage status information into the usage status information recording unit 408c, the usage status information being an endoscope withdrawal time at which the distal end portion 24 of the endoscope 2 was withdrawn from the inside of the body of the subject.

Subsequently, the control unit 410 stops: imaging by means of the endoscope 2; recording image data captured by the endoscope 2; detection of whether or not insertion into the subject has been detected by the image diagnosis apparatus 5; and region detection for internal organs and regions by means of the image diagnosis apparatus 5 (Step S215). After Step S215, the control apparatus 4 ends the process and returns to a main routine in FIG. 5.

At Step S216, the determination unit 410c determines whether or not the operating surgeon has performed an operation on the operating portion 22. In a case where the determination unit 410c determines that the operating surgeon has performed an operation on the operating portion 22 (Step S216: Yes), the control apparatus 4 proceeds to Step S217 described later. On the contrary, in a case where the determination unit 410c determines that the operating surgeon has not operated the operating portion 22 (Step S216: No), the control apparatus 4 proceeds to Step S220 described later.

At Step S217, the determination unit 410c determines whether or not the operation performed by the operating surgeon was an imaging operation. Specifically, in a case where a release signal has been input from the operating portion 22, the determination unit 410c determines that the operating surgeon has performed the imaging operation on the operating portion 22. In a case where the control unit 410 determines that the operating surgeon has performed the imaging operation on the operating portion 22 (Step S217: Yes), the control apparatus 4 proceeds to Step S218 described later. On the contrary, in a case where the determination unit 410c determines that the operating surgeon has not performed the imaging operation on the operating portion 22 (Step S217: No), the control apparatus 4 proceeds to Step S219 described later.

At Step S218, the imaging control unit 410a causes the imaging element 203 to capture a still image (still image data). Specifically, the imaging control unit 410a records a still image file into the image data recording unit 408b, the still image file including, in association with each other, the still image data generated by the imaging element 203 and the imaging time at which the still image data were captured by the imaging element 203. The communication control unit 410e then outputs the still image file to the medical assistance apparatus 6 via the network N100. The still image data correspond to an image higher in resolution (having a larger number of pixels) than live view image data (for example, a 4K image). After Step S218, the control apparatus 4 proceeds to Step S220.

At Step S219, the acquisition unit 410b acquires operation information on the operation performed on the operating portion 22 by the operating surgeon and records the operation information in the usage status information recording unit 408c. The operation may be a water feeding operation for the endoscope 2 or a change operation for changing the angle of view of the imaging optical system 202. The operation may of course be an operation for treatment using an energy device not illustrated in the figure. After Step S219, the control apparatus 4 proceeds to Step S220.

At Step S220, the determination unit 410c determines whether or not a predetermined time period (for example, five minutes) has elapsed since the endoscope 2 started imaging. In a case where the determination unit 410c determines that the predetermined time period has elapsed since the endoscope 2 started imaging (Step S220:

Yes), the control apparatus 4 proceeds to Step S215. This situation is assumed to indicate that some abnormality has occurred in the subject. For example, the situation may be: a situation where the endoscope 2 is unable to be inserted due to a health problem in the subject; a case where the endoscope 2 has not been cleaned; or imaging associated with maintenance or activation check for the endoscope 2. In this case, the acquisition unit 410b records usage status information that is information indicating that the endoscope 2 has not been inserted in the subject, into the usage status information recording unit 408c. On the contrary, in a case where the determination unit 410c determines that the predetermined time period has not elapsed since the endoscope 2 started imaging (Step S220: No), the control apparatus 4 returns to Step S202.

### Details of Report Preparation Process

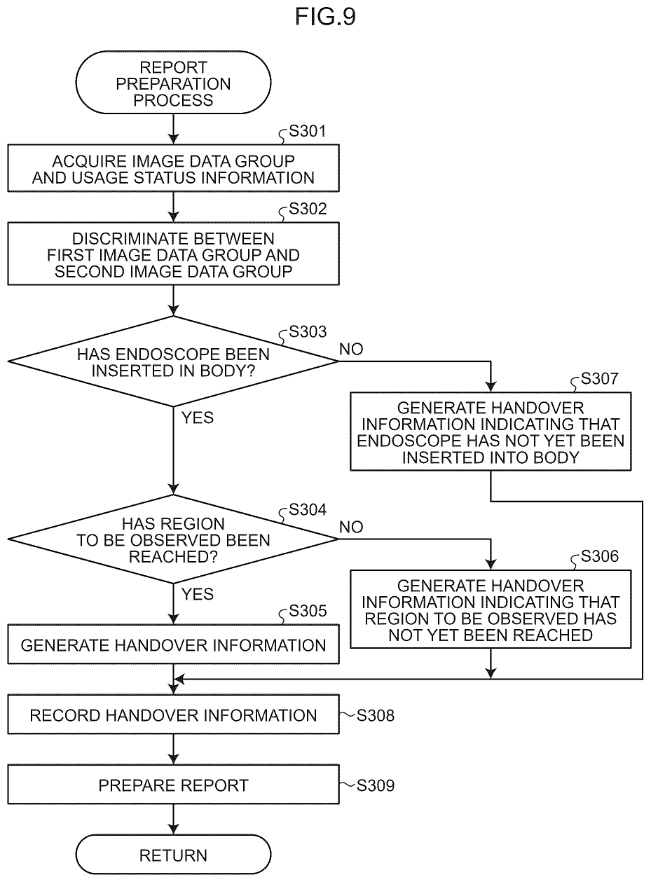

Details of the report preparation process at Step S108 in FIG. 5 will be described next. FIG. 9 is a flowchart illustrating an outline of the report preparation process.

As illustrated in FIG. 9, the acquisition unit 661 acquires an image data group and usage status information from the control apparatus 4 via the network N100 (Step S301).

Subsequently, on the basis of the image data group and usage status information acquired by the acquisition unit 661, the discrimination unit 662 discriminates between a first image data group captured upon observation of a predetermined region to be observed and a second image data group captured after the observation of the predetermined region to be observed (Step S302).

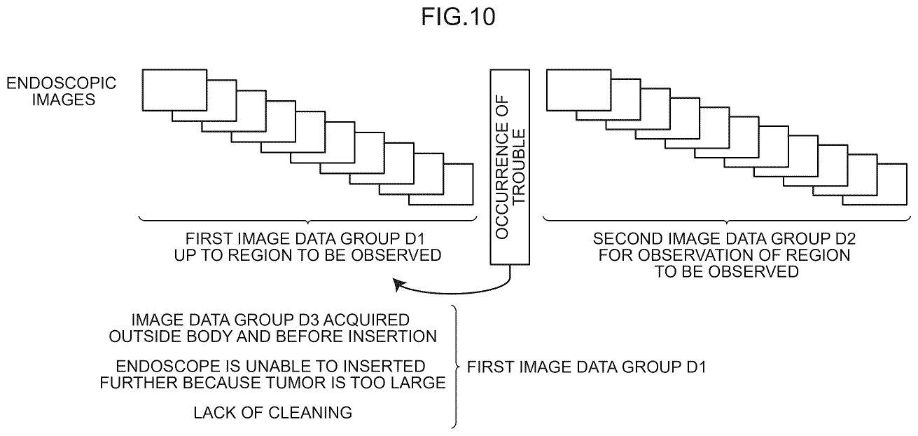

FIG. 10 is a diagram schematically illustrating an outline of how the discrimination unit 662 discriminates the group of image data into the first image data group and the second image data group.

The endoscope 2 implements imaging to generate a first image data group D1 including multiple sets of image data until the distal end portion 24 reaches a region to be observed by the operating surgeon inserting the distal end portion 24 in the body of the subject, and implements imaging to generate a second image data group D2 including multiple sets of image data also after reaching the region to be observed. In this case, for insertion of the distal end portion 24 of the endoscope 2 (lower endoscope) in the body of the subject from the outside of the body, the operating surgeon wants to insert the distal end portion 24 to the predetermined region to be observed as promptly as possible to reduce discomfort for the subject, but some kind of trouble may occur, such as a health problem in the subject who is a patient, obstruction disabling further insertion due to a large tumor in the lumen of the subject, or the endoscope 2 not having been cleaned yet. Furthermore, the first image data group D1 includes an image data group D3 acquired outside the body and before the insertion, the image data group D3 corresponding to a period up to the insertion of the endoscope 2 in the body of the subject and being due to a health problem in the subject or the endoscope 2 not having been cleaned yet.

As illustrated in FIG. 10, the discrimination unit 662 thus discriminates between the first image data group D1 and the second image data group D2, on the basis of: the image data group acquired by the acquisition unit 661; and cleaning history information, information indicating whether or not there has been insertion in the body, and detection result information on internal organs and regions, which are included in the usage status information.

By reference back to FIG. 9, description of the process from Step S303 will now be resumed.

At Step S303, on the basis of the first image data group discriminated by the discrimination unit 662 and the usage status information, the determination unit 663 determines whether or not the distal end portion 24 of the endoscope 2 has been inserted in the body of the subject. In a case where the determination unit 663 determines that the distal end portion 24 of the endoscope 2 has been inserted in the body of the subject (Step S303: Yes), the medical assistance apparatus 6 proceeds to Step S304 described later. On the contrary, in a case where the determination unit 663 determines that the distal end portion 24 of the endoscope 2 has not been inserted in the body of the subject (Step S303: No), the medical assistance apparatus 6 proceeds to Step S307 described later.

At Step S304, on the basis of the usage status information, the determination unit 663 determines whether or not the distal end portion 24 of the endoscope 2 has reached a preset region to be observed. Specifically, on the basis of the detection result information detected by the image diagnosis apparatus 5 and the observation order table W1 (see the pattern Q1 in FIG. 6) included in the usage status information, the determination unit 663 determines whether or not the distal end portion 24 of the endoscope 2 has reached the preset region to be observed. For example, in a case where the deepest internal organ or region is the “ileum” and the position in the observation order is “9” in the observation order table W1, and the detection result from the image diagnosis apparatus 5 includes the “ileum”, the determination unit 663 determines that the distal end portion 24 of the endoscope 2 has reached the preset region to be observed. In a case where the determination unit 663 determines that the distal end portion 24 of the endoscope 2 has reached the preset region to be observed (Step S304: Yes), the medical assistance apparatus 6 proceeds to Step S305 described later. On the contrary, in a case where the determination unit 663 determines that the distal end portion 24 of the endoscope 2 has not reached the preset region to be observed (Step S304: No), the medical assistance apparatus 6 proceeds to Step S306 described later.

At Step S305, on the basis of the second image data group and the usage status information, the generation unit 664 generates handover information and records the handover information into the handover information DB 647.

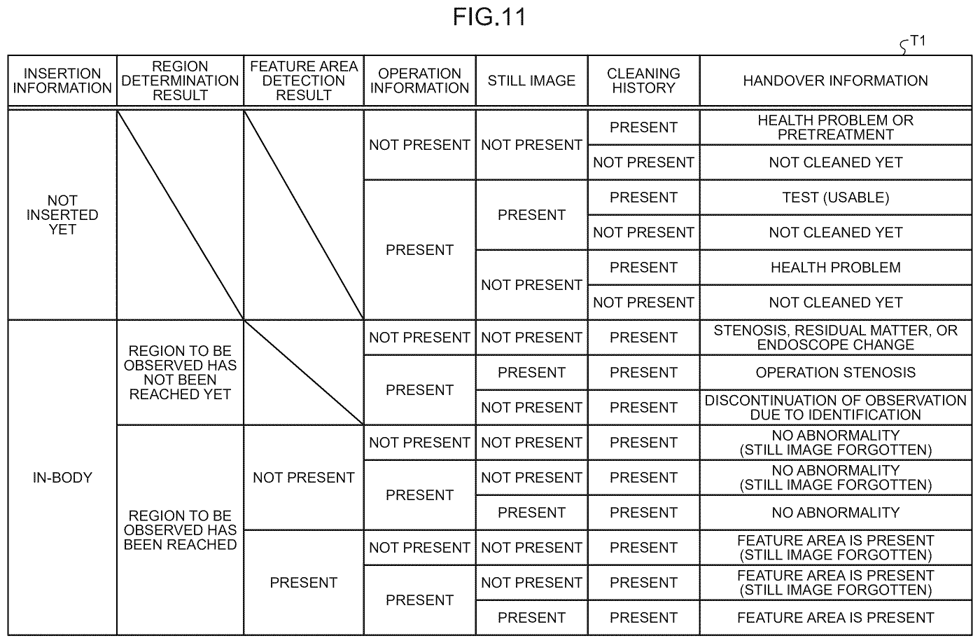

FIG. 11 is a diagram illustrating an example of a handover information table generated by the generation unit 664, the handover information table including each condition and handover information in association with each other. A handover information table T1 illustrated in FIG. 11 has, recorded therein, each condition and handover information in association with each other.

Specifically, the generation unit 664 refers to the handover information table T1, and generates the handover information on the basis of detection results detected by the image diagnosis apparatus 5 and included in usage history information, the second image data group, insertion information, operation information, and cleaning information. For example, in a case where insertion information in the detection results is “in-body”, the region determination result is “region to be observed has been reached”, a feature area detection result is “present”, operation information is “present”, and a still image is “present”, the generation unit 664 generates handover information, “feature area is present”, indicating that the subject has a lesion, such as a tumor.

Furthermore, in a case where the insertion information in the detection results is “in-body”, the region determination result is “region to be observed has been reached”, a feature area detection result is “not present”, operation information is “not present”, and a still image is “not present”, the generation unit 664 generates handover information, “no abnormality (still image forgotten (still image not captured))” indicating that the subject has no abnormality.

As described above, the generation unit 664 refers to the handover information table T1, and generates the handover information on the basis of the second image data group included in the usage history information, the detection results detected by the image diagnosis apparatus 5, the insertion information, the operation information, and the cleaning information. After Step S305, the medical assistance apparatus 6 proceeds to Step S308 described later.

At Step S306, on the basis of the second image data group and the usage status information, the generation unit 664 generates handover information. In this case, the generation unit 664 refers to the handover information table T1 in FIG. 11 and generates the handover information on the basis of the second image data group and the usage status information. For example, in a case where the insertion information in the detection results is “in-body”, the region determination result is “region to be observed has not been reached yet”, operation information is “present”, and a still image is “present”, the generation unit 664 generates “stenosis” as the handover information indicating that the subject has a stenosis due to a tumor, for example. After Step S307, the medical assistance apparatus 6 proceeds to Step S308 described later.

At Step S307, the generation unit 664 refers to the handover information table T1 in FIG. 11, and on the basis of the first image data group and the usage status information, the generation unit 664 generates handover information. For example, in a case where the insertion information in the detection results is “not inserted yet”, operation information is “present, a still image is “present”, and a cleaning history is “present”, the generation unit 664 generates, as the handover information, “test (usable)” indicating a test or maintenance by an operating surgeon or a technician who maintains the endoscope. Furthermore, in a case where the insertion information in the detection results is “not inserted yet”, operation information is “not present”, a still image is “not present”, and a cleaning history is “present”, the generation unit 664 generates, as the handover information, “health problem or pretreatment” indicating a health problem in the subject or an allergy due to pretreatment. Furthermore, in a case where the insertion information in the detection results is “not inserted yet”, operation information is “present”, a still image is “not present”, and a cleaning history is “not present”, the generation unit 664 generates, as the handover information, “not cleaned yet”, indicating that the endoscope 2 has not been cleaned yet. After Step S307, the medical assistance apparatus 6 proceeds to Step S308 described later.

At Step S308, the recording control unit 665 records the handover information generated by the generation unit 664, into the handover information DB 647. In this case, the recording control unit 665 records the handover information in association with patient information or operating surgeon information, into the handover information DB 647.

Subsequently, on the basis of operation information from the input unit 63 or the laptop personal computer 7, an image data group and usage status information acquired by the acquisition unit 661, patient information, and handover information generated by the generation unit 664, the preparation unit 666 performs report preparation for preparing report information on a subject (Step S309). Specifically, on the basis of the operation information and the usage status information, the preparation unit 666 prepares report information (an examination report) including a determination result for the region to be reached by the distal end portion 24 of the endoscope 2 and handover information for a conference for the subject, and records this report information in association with the patient information or operating surgeon information, into the report information DB 648. After Step S308, the medical assistance apparatus 6 returns to the main routine in FIG. 5.

### Outline of Process for Examination Yet to be Completed Details of a process for an examination yet to be completed at Step S114 in FIG. 5 will be described next.

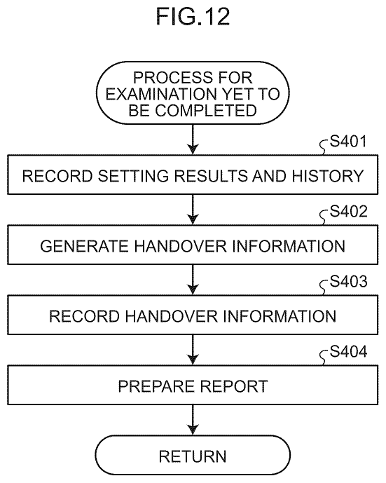

FIG. 12 is a flowchart illustrating an outline of the process for an examination yet to be completed.

As illustrated in FIG. 12, on the basis of what is input from the laptop personal computer 7 or control apparatus 4, the preparation unit 666 records setting results for settings for a subject to be observed and the patient name of the subject and a history of how the subject came to remain unobserved, into the patient information DB 641 (Step S401). Specifically, how the subject came to remain unobserved is, for example, discontinuation due to a sudden change caused by deterioration in physical condition of the subject, discontinuation due to an allergic reaction to a pre-procedure injection, incompatibility of the type of the endoscope 2 with the subject (patient), lack of maintenance of the endoscope 2, failure of the endoscope 2, or the endoscope 2 not having been cleaned.

Subsequently, the generation unit 664 refers to the handover information table T1 in FIG. 11 and generates handover information on the basis of a first image data group and usage status information (Step S402). For example, in a case where the insertion information in the detection results is “not inserted yet”, operation information is “not present”, a still image is “not present”, and a cleaning history is “present”, the generation unit 664 generates, as the handover information, “health problem or pretreatment”, indicating a health problem in the subject or an allergy due to pretreatment.

Thereafter, the recording control unit 665 records the handover information generated by the generation unit 664 into the handover information DB 647 (Step S403).

Subsequently, on the basis of operation information from the input unit 63 or laptop personal computer 7 and handover information on how the patient came to remain unobserved with the endoscope 2, the preparation unit 666 prepares report information on how the subject came to remain unobserved with the endoscope 2, and records this report information in association with patient information or operating surgeon information into the report information DB 648 (Step S404). When an operating surgeon performs image interpretation using the report information on the patient next time or later, the operating surgeon is thereby able to know how the patient came to remain unobserved. After Step S402, the medical assistance apparatus 6 returns to the main routine in FIG. 5.

According to one embodiment described above, on the basis of a determination result determined by the determination unit 663, the determination result being on whether or not the endoscope 2 has reached a region to be observed, and usage status information acquired by the acquisition unit 661, the generation unit 664 generates handover information for a report related to observation of the region to be observed in a patient, and the recording control unit 665 records the handover information generated by the generation unit 664 into the handover information DB 647, and useful medical information is thus able to be handed over according to a usage status of the endoscope 2.

Furthermore, according to one embodiment, in a case where the determination unit 663 determines that the endoscope 2 has not been inserted in the body of the subject, the generation unit 664 generates handover information indicating that the endoscope 2 fell short of being inserted in the body of the subject, and clinical data helpful in subsequent observation of the subject are thus able to be saved.

Furthermore, according to one embodiment, in a case where the determination unit 663 determines that the endoscope 2 has not been inserted in the body of the subject, the generation unit 664 generates, on the basis of operation information included in the usage status information, handover information indicating that the endoscope 2 fell short of being inserted in the body of the subject, and information indicating whether or not the usage was temporary due to imaging for maintenance is thus able to be saved.

Furthermore, according to one embodiment, on the basis of the operation information and cleaning history information included in the usage status information, the generation unit 664 generates handover information indicating that the endoscope 2 fell short of being inserted in the body of the subject, and information indicating that the endoscope 2 has not been cleaned yet is thus able to be saved.

Furthermore, according to one embodiment, in a case where the determination unit 663 determines that the endoscope 2 has not reached the region to be observed, the generation unit 664 generates handover information indicating that the endoscope 2 fell short of being inserted up to the region to be observed, and medical information indicating that insertion failed due to the subject's condition, for example, a stenosis or residual matter, is thus able to be saved.

Furthermore, according to one embodiment, when a feature area in the subject is determined to have been detected in a case where the determination unit 663 determines that the endoscope 2 has reached the region to be observed, the generation unit 664 generates handover information indicating that an abnormality has occurred in the subject, and when a feature area in the subject is determined to have not been detected in the case where the determination unit 663 determines that the endoscope 2 has reached the region to be observed, the generation unit 664 generates handover information indicating that the subject is normal, and medical information is thus able to be saved.

Furthermore, according to one embodiment, in a case where the determination unit 663 determines that a still image is present, the generation unit 664 generates handover information indicating that the still image is present, and medical information related to a tumor or a feature area in the subject is thus able to be used in a conference, for example.

Various embodiments may be formed by combination, as appropriate, of more than one component disclosed with respect to the above described medical assistance system according to the embodiment of the present disclosure. For example, some of all of the components described with respect to the medical assistance system according to the above described embodiment of the present disclosure may be eliminated. Furthermore, any components described with respect to the medical assistance system according to the above described embodiment of the present disclosure may be combined as appropriate.

Furthermore, any “unit” described above with respect to the medical assistance system according to the embodiment of the present disclosure may be read, for example, as a “means” or a “circuit”. For example, a control unit may be read as a control means or a control circuit.

Furthermore, programs to be executed by the medical assistance system according to the embodiment of the present disclosure may be provided by being recorded as file data in an installable format or executable format in a computer-readable recording medium, such as a CD-ROM, a flexible disk (FD), a CD-R, a digital versatile disk (DVD), a USB medium, or a flash memory.

Furthermore, the programs to be executed by the medical assistance system according to the embodiment of the present disclosure may be configured to be stored on a computer connected to a network, such as the Internet, and to be provided by being downloaded via the network.

In the description of the flowcharts in this specification, the order of the steps in each process is disclosed by use of expressions, such as “firstly”, “thereafter”, and “subsequently”, but the order in each process needed for implementation of the disclosure is not uniquely defined by these expressions. That is, the orders of the processes in the flowcharts described in this specification may be modified so long as no contradiction arises from that modification. Without being limited to such programs consisting of simple branching processes, a larger number of decision items may be comprehensively determined and branched.

An effect provided according to the present disclosure is that useful medical information is able to be handed over according to a usage status of an endoscope.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical assistance apparatus comprising a processor comprising hardware, the processor being configured to:
acquire usage status information related to a usage status of an endoscope from a time point at which the endoscope is outside a body of a subject to a time point at which the endoscope observes a desired region to be observed in a body cavity of the subject;
determine, based on the usage status information, whether or not the endoscope has been inserted into the body cavity or whether or not the endoscope has reached the desired region to be observed;
when it is determined that the endoscope has not been inserted into the body cavity or that the endoscope has not reached the desired region to be observed, generate handover information including each condition and handover content in association with each other for a report related to an observation of the desired region to be observed, the report indicating that the endoscope fell short of being inserted up to the desired region to be observed; and
record the handover information.

2. The medical assistance apparatus according to claim 1, wherein
the usage status information includes insertion information indicating whether or not the endoscope has been inserted into the body of the subject and being based on an image data group captured by the endoscope, and
the processor is configured to
determine, based on the insertion information, whether or not the endoscope has been inserted into the body of the subject, and
when it is determined that the endoscope has not been inserted into the body of the subject, generate the handover information indicating that the endoscope fell short of being inserted into the body of the subject.

3. The medical assistance apparatus according to claim 2, wherein
the usage status information further includes operation information on an operation by a user on an operating portion included in the endoscope, and
when it is determined that the endoscope has not been inserted into the body of the subject, the processor is configured to generate, based on the operation information, the handover information indicating that the endoscope fell short of being inserted into the body of the subject.

4. The medical assistance apparatus according to claim 3, wherein
the usage status information further includes cleaning history information related to a cleaning history of the endoscope, and
the processor is configured to generate the handover information indicating that the endoscope fell short of being inserted into the body of the subject based on the operation information and the cleaning history information.

5. The medical assistance apparatus according to claim 4, wherein
the usage status information further includes feature area detection information indicating whether or not a feature area in the body of the subject has been detected and being based on the image data group captured by the endoscope, and
the processor is configured to
when it is determined that the endoscope has reached the desired region to be observed, determine, based on the feature area detection information, whether or not the feature area has been detected,
when it is determined that the endoscope has reached the desired region to be observed and that the feature area has been detected, generate the handover information indicating that an abnormality has occurred in the subject, and
when it is determined that the endoscope has reached the desired region to be observed and that the feature area has not been detected, generate the handover information indicating that the subject is normal.

6. The medical assistance apparatus according to claim 5, wherein
the usage status information further includes release information related to a history of a release signal from an operating part included in the endoscope, and
the processor is configured to
determine, based on the release information, whether or not a still image is present, and
when it is determined that the still image is present, generate the handover information indicating that the still image is present.

7. The medical assistance apparatus according to claim 6, wherein
the processor is further configured to
acquire the image data group generated by the endoscope continuously imaging,
discriminate the image data group into a first image data group for up to an insertion of the endoscope in the body of the subject and a second image data group for after the insertion of the endoscope in the body of the subject, and
the usage status information further includes detection result information on detection of multiple internal organs and regions in the subject, the detection being based on the second image data group.

8. A medical assistance system, comprising:
an endoscope;
a control apparatus configured to control the endoscope;
an image diagnosis apparatus; and
a medical assistance apparatus, wherein
the endoscope comprises:
an insertion portion configured to be inserted into a subject; and
a distal end portion that is provided at a distal end of the insertion portion, the distal end portion being configured to successively generate an image data group by continuously imaging multiple regions to be observed that have been preset for the subject, and output the image data group to the control apparatus, and
the image diagnosis apparatus is configured to
generate detection result information obtained by detecting each of the multiple regions to be observed from the image data group by using the image data group and an inference model that has been pretrained with features of internal organs and regions, and
output the detection result information to the control apparatus,
the medical assistance apparatus comprises a processor comprising hardware, and
the processor is configured to
acquire usage status information related to a usage status of the endoscope up to observation of a region to be observed predetermined for the subject,
determine, based on the usage status information, whether or not the endoscope has reached the region to be observed,
generate, based on a determination result on whether or not the endoscope has reached the region to be observed and on the usage status information, handover information for a report related to an observation of the region to be observed, and
record the handover information.

9. The medical assistance system according to claim 8, wherein
the usage status information includes insertion information indicating whether or not the endoscope has been inserted into a body of the subject and being based on the image data group captured by the endoscope, and
the processor is configured to
determine, based on the insertion information, whether or not the endoscope has been inserted into the body of the subject, and
when it is determined that the endoscope has not been inserted into the body of the subject, generate the handover information indicating that the endoscope fell short of being inserted into the body of the subject.

10. The medical assistance system according to claim 8, wherein
the usage status information includes operation information on an operation by a user on an operating portion included in the endoscope, and
when it is determined that the endoscope has not been inserted into a body of the subject, the processor is configured to generate, based on the operation information, the handover information indicating that the endoscope fell short of being inserted into the body of the subject.

11. The medical assistance system according to claim 8, wherein
the usage status information includes cleaning history information related to a cleaning history of the endoscope, and
the processor is configured to generate the handover information indicating that the endoscope fell short of being inserted into a body of the subject based on the operation information and the cleaning history information.

12. The medical assistance system according to claim 8, wherein
the usage status information includes feature area detection information indicating whether or not a feature area in a body of the subject has been detected and being based on the image data group captured by the endoscope, and
the processor is configured to
when it is determined that the endoscope has reached the desired region to be observed, determine, based on the feature area detection information, whether or not the feature area has been detected,
when it is determined that the endoscope has reached the desired region to be observed and that the feature area has been detected, generate the handover information indicating that an abnormality has occurred in the subject, and
when it is determined that the endoscope has reached the desired region to be observed and that the feature area has not been detected, generate the handover information indicating that the subject is normal.

13. The medical assistance system according to claim 8, wherein
the usage status information includes release information related to a history of a release signal from an operating part included in the endoscope, and
the processor is configured to
determine, based on the release information, whether or not a still image is present, and
when it is determined that the still image is present, generate the handover information indicating that the still image is present.

14. The medical assistance system according to claim 8, wherein
the processor is further configured to
acquire the image data group generated by the endoscope continuously imaging,
discriminate the image data group into a first image data group for up to an insertion of the endoscope in a body of the subject and a second image data group for after the insertion of the endoscope in the body of the subject, and
the detection result information is based on the second image data group.

15. A method of operating a medical assistance apparatus, the method comprising:
acquiring usage status information related to a usage status of an endoscope from a time point at which the endoscope is outside a body of a subject to a time point at which the endoscope observes a desired region to be observed in a body cavity of the subject;
determining, based on the usage status information, whether or not the endoscope has reached the desired region to be observed;
when it is determined that the endoscope has not reached the desired region to be observed based on a determination result on whether or not the endoscope has reached the desired region to be observed and on the usage status information, generating handover information including each condition and handover content in association with each other for a report related to an observation of the desired region to be observed, the report indicating that the endoscope fell short of being inserted up to the desired region to be observed; and
recording the handover information.

16. The method according to claim 15, wherein
the usage status information includes insertion information indicating whether or not the endoscope has been inserted into the body of the subject and being based on an image data group captured by the endoscope, and
based on the insertion information, whether or not the endoscope has been inserted into the body of the subject is determined, and
when it is determined that the endoscope has not been inserted into the body of the subject, the handover information indicating that the endoscope fell short of being inserted into the body of the subject is generated.

17. The method according to claim 15, wherein
the usage status information includes operation information on an operation by a user on an operating portion included in the endoscope, and
when it is determined that the endoscope has not been inserted in the body of the subject, the handover information indicating that the endoscope fell short of being inserted into the body of the subject is generated based on the operation information.

18. The method according to claim 15, wherein
the usage status information includes cleaning history information related to a cleaning history of the endoscope, and
the handover information indicating that the endoscope fell short of being inserted into the body of the subject is generated based on the operation information and the cleaning history information.

19. The method according to claim 15, wherein
the usage status information includes feature area detection information indicating whether or not a feature area in the body of the subject has been detected and being based on an image data group captured by the endoscope,
when it is determined that the endoscope has reached the desired region to be observed, whether or not the feature area has been detected is determined based on the feature area detection information, and
when it is determined that the endoscope has reached the desired region to be observed and that the feature area has been detected, the handover information indicating that an abnormality has occurred in the subject is generated,
when it is determined that the endoscope has reached the desired region to be observed and that the feature area has not been detected, the handover information indicating that the subject is normal is generated.

20. The method according to claim 15, wherein
the usage status information includes release information related to a history of a release signal from an operating part included in the endoscope, and
whether or not a still image is present is determined based on the release information, and
when it is determined that the still image is present, the handover information indicating that the still image is present is generated.
